(19) United States
(12) Patent Application Publication
(10) Pub. No.: US 2026/0103493 A1
(43) Pub. Date: Apr. 16, 2026

(54) FUNCTIONAL NATIVE POTATO PROTEIN, AND METHOD FOR PRODUCING SAME

(71) Applicant: EMSLAND STÄRKE GMBH, Emlichheim (DE)

(72) Inventors: Nico REINS, Wilsum (DE); Fabian GRUBER, Lingen (DE)

(21) Appl. No.: 19/117,136

(22) PCT Filed: Sep. 27, 2023
(86) PCT No.: PCT/DE2023/100725

§ 371 Date: Mar. 31, 2025

(30) Foreign Application Priority Data
Sep. 30, 2022 (DE) 20 2022 105 549.1

(51) Int. Cl. C07K 14/415; A23J 1/00; A23J 1/14

(52) U.S. Cl. CPC C07K 14/415; A23J 1/006; A23J 1/14

(57) ABSTRACT

The invention relates to a method for the preparation of potato protein and a native functional potato protein with a molecular weight of 10-120 kDa (according to SDS-Page primary structure), producible from potato amniotic fluid, which is obtained by the mechanical solid/liquid separation of chopped, cleaned potato parts, comprising: a) Protein content of at least 84% by weight absolute dry; b) a humidity content as dried protein: max. 10% by weight; c) Product solubility/protein solubility in water of 75-100%; d) Ash content of max. 3% by weight, which can be produced by a) chopping the potato raw material; b) mechanical separation of the potato mash into solid phase and liquid phase (potato amniotic fluid); c) pH adjustment to 6-9; d) ultrafiltration of the liquid phase; d) diafiltration of the ultrafiltration retentate; e) optionally before or after step d) removal of undesirable flavors by adsorption techniques; f) sterilizing the resulting protein solution; g) optionally drying the protein solution.

### BACKGROUND OF THE INVENTION

### Field of the Invention

The invention relates to a potato protein with a molecular weight of 10-120 kDa (according to SDS-Page primary structure), and a method for its preparation and use in foods.

### Description of Related Art

The potato amniotic fluid produced during the chopping of cleaned potatoes, for example during potato starch production, poses a challenge for the industry, as it cannot be disposed of in sewage treatment plants or discharged into bodies of water without further treatment. Separation of the proteins contained (approx. 2%) is absolutely necessary. Precipitation often takes place using a strong temperature increase to >90° C. and/or pH shift and subsequent isolation of the coagulated proteins by mechanical separation. The proteins obtained in this way no longer have any functional properties (no functionality, no solubility, no gelation or foam formation), as the tertiary structure of the proteins is irreversibly destroyed during thermal coagulation. In addition, protein fractions isolated in this way have a strong off-flavor and often a high proportion of antinutritive substances, in particular a high glycoalkaloid content. All this makes their use in the food industry unattractive and, due to their high glycoalkaloid content, also prohibited by law.

The use of plant proteins in the human diet is becoming increasingly important for a variety of reasons. Vegetarian and vegan diets are becoming increasingly widespread, and the use of animal proteins is sometimes undesirable for religious, ethical or health reasons (e.g. allergies to milk proteins, etc.). Animal proteins also have a shorter shelf life when moist. However, plant proteins often have the disadvantage that they are not complete in terms of their amino acid spectrum and therefore have a lower biological value. Potato protein has one of the best nutritional values among plant proteins, a high nutrient content, high digestibility, and a balanced amino acid spectrum that is comparable to milk and egg protein. The high lysine content in potato protein makes it a good substitute for lysine-poor proteins such as those from cereals.

The textbook “Advances in Potato Chemistry and Technology”, Elsevier Inc. 2016, which is in its 2^{nd} edition and will apparently be published in its third edition in 2024 due to high demand, is to be regarded as the generally known state of the art on potato proteins. Chapter 4, pp. 75-104, Potato Proteins, “Functional food Ingredients” of this standard work shows that potato protein-which here refers to the proteins found in potato tubers-consists of three main groups: Patatin (MW: 40-45 kDa—as well as a dimer of approx. 88 kDa, represents approx. 40% by weight of the soluble tuber protein), the protease inhibitors (MW: 7-21 kDa, approx. 50% by weight of the soluble tuber protein) and higher molecular weight proteins (MW>40 kDa, approx. 10% by weight of the soluble tuber protein).

Patatin, also known as tuberin, is therefore a group of glycoproteins and exhibits lipid acyl hydroxygenase activity. It has an isoelectric point of approx. 4.9. It is temperature-sensitive and irreversibly loses its tertiary structure from 45° C. and the alpha-helical part of patatin denatures from 55° C. (see p. 76, 2.2. “Patatin”). Precipitation in acid has a similar effect. It has the same biological value as egg albumin or lysozyme, has antioxidant properties and better emulsifying properties than soy protein. In contrast to most vegetable proteins, Patatin has a high proportion of the essential amino acid lysine and sulphur-containing amino acids. It should be noted that the various protein groups are unevenly distributed in the tuber-many valuable components only occur in the outer area under the husks (see G. Barel, I. Ginzberg, J. Exp. Bot. 2008, 59, 3347-3357). In addition, many elements such as potassium, calcium, magnesium, iron, zinc, manganese and copper cations are more concentrated in the potato skin (see p. 116, Chapter 5: “Potato Proteins, Lipids, and Minerals” by S. O. Kärenlampi, P. J. White, from “Advances in Potato Chemistry and Technology”).

Protease inhibitors, also known as tuberinin, are storage proteins. The higher-molecular proteins of this very diverse group contain lectins, phenol oxidases and lipoxygenases and some have peptidase or carboxypeptidase inhibitor properties.

It should be noted that all stated values and the analytical values are subject to fluctuation, as is inevitable with natural products, and that aggregates and compounds with a higher molecular weight may also be present in small quantities that are not visible in the SDS gel. The SDS gel chromatogram only shows larger quantities-small quantities do not produce any bands. In addition, only qualitative conclusions can be drawn about the sample composition, not quantitative ones.

Low to non-denatured potato protein has good functional properties such as solubility in aqueous and alcoholic solutions (globulins dissolve in alcoholic solutions), gel and foam formation, as well as good emulsifying properties, which makes it attractive for use in foods in addition to its nutritional properties. The functional properties are strongly influenced by the production conditions of the protein (heat, pH value, salts, shear forces, stress), as these can lead to denaturation and thus loss of functionality. As explained on p. 87 of “Advances in Potato Chemistry and Technology” under 2.3.3. “Foaming Properties”, ultrafiltration can be used to improve the foaming properties of potato protein (see Koenigsveld 2002). “Potato Chemisty” also explains that high mechanical stress leads to denaturation of potato protein.

In “Applied Food Protein Chemistry” (edited by Z. Ustunol, Chapter 4: “Physical, Chemical and Processing-induced Changes in Proteins”, 2015, 1^{st} Edition, Wiley Blackwell), the fundamental effects of physical and chemical influences on the protein and its functionalities are described in great detail. Z. Ustunol is a professor at the University of Michigan, which means that this information is passed on to students of food technology and is the knowledge of the person skilled in the art. Denaturation means changes in the tertiary and secondary structure of a protein that lead to reversible or irreversible unfolding of the protein. Here, the inter- and intramolecular interactions are broken by external factors. Heat reduces the stability of non-covalent interactions and breaks bonds such as hydrogen bonds. The humidity of the protein also influences its stability to heat. A hydrated protein is more mobile and can unfold more easily, which is why it is also less stable to heat denaturation.

Damodaran already described in 2008 (see “Amino Acids, peptides and proteins” in Fennema's Food Chemistry, 2008, 5^{th} Edition, CRC Books) that heat coagulation is an irreversible denaturation for most proteins. High pressures also lead to the unfolding of proteins, as native proteins have cavities that are not present in the denatured (unfolded) state. In addition, Bianco 2012 (V. Bianco, S. Iskrov, G. Franzese, J. Biol. Phys. 2012, 38, 27-48) described that high pressures can force water into the cavities of the proteins, which leads to swelling and denaturation of the protein. Similarly, mechanical shear forces (e.g. centrifuging, shaking, mixing, whipping, etc.) can also lead to denaturation.

In addition to the physical means mentioned, proteins can also be chemically denatured. One common way is to adjust the pH value. The isoelectric point of a protein is the pH value at which the total charge of the protein is zero. This minimizes the repulsive forces of the charges, which is why the proteins can aggregate and coagulate, so that their solubility is lowest at the isoelectric point. At pH values below and above this value, the solubility of the protein increases again. Extreme pH conditions, which lead to high positive or negative charge of the protein, can also lead to strong intramolecular repulsion and thus denaturation (see S. Damodaran, “Amino Acids, peptides and proteins” in Fennema's Food Chemistry, 2008, 5^{th} Edition, CRC Books; J. Culbertson, “Proteins functional properties” in Food Chemistry: Principles and Applications, 2012, 3^{rd} Edition, Science Technology).

The electrostatic interactions can also be used to precipitate proteins using organic solvents that are less polar than water. This effect can be utilized for the precipitation of proteins in extractions or for purification (Z. Ustunol, 4.2.2.2 Organic-solvent-induced denaturation in “Applied Food Protein Chemistry” 2015, 1^{st} edition, Wiley Blackwell).

The addition of salts also has a strong influence on protein properties. Ustunol describes that protein solubility is increased in dilute salt solutions of low ionic strength (<0.2 M) regardless of the type of salt. However, the opposite effect occurs at high salt concentrations (>1 M). The salt interacts with the water so that protein-protein interactions are intensified, which leads to precipitation of the proteins. The salt concentration at which precipitation occurs depends on the type of protein.

Osborne was able to demonstrate these effects as early as 1905 (see T. B. Osborne, G. F. Campbell, J. Am. Chem. Soc. 1896, 18, 575-582; T. B. Osborne, I. F. Harris, Am. J. Phys. 1905, 13, 35-44 and T. B. Osborne, “Die Pflanzenproteine” in Ergebnisse der Physiologie 1910, 10, 47-215) to obtain the so-called Osborne fractions from cereal protein, which could be obtained according to their different solubilities: While albumins can be extracted with water, globulins can be extracted with saline solutions. Prolamins can be extracted with ethanol (70%). Glutelins, on the other hand, remain in arrears. The designation of these so-called Osborne fractions has become established in the entire field of plant proteins.

For these reasons, the starting material (with skin), the preparation method of the protein and the conditions of further processing into a food product have an immense influence on the condition, functionality and composition of the protein obtained. All processing steps therefore have an impact on the properties of the final protein product. All methods associated with heating, such as pasteurization, drying, sterilizing, blanching, cooking and others, result in complete or partial denaturation of the protein, which occurs at the temperatures as low as 40° C.

Due to the attractive properties of potato protein and the increased demand for proteins already mentioned at the beginning, a large number of different processes for the preparation of potato protein are now known. The first patents for the production of potato protein were filed back in 1997. A number of scientific publications have also been published on this topic in the form of review articles describing the most common preparation methods and citing advantages and disadvantages (see for example: Y. Fu, W.-N. Liu, O. P. Soladoye, Int. J. Food Sci. Technol. 2020, 55, 2314-2322 or S. LØkra, K. O. Straetkvern, Food 2009, Special Issue 1, 88-95). Basically, the methods can be traced back to three main methods: 1) Precipitation/coagulation—by means of temperature adjustment and/or pH value adjustment, addition of a precipitant or complexation; 2) Membrane separation; 3) Chromatography. The starting material is mainly potato amniotic fluid, which is a by-product of the starch industry and contains approx. 1-2% potato protein.

In industry, the proteins contained in potato amniotic fluid are often isolated by coagulation by adjusting the temperature to >60° C. and/or adjusting the pH value to <6 and subsequent mechanical separation of the coagulated protein, which, as stated in “Advances in Potato Chemistry and Technology”, is extremely detrimental to the properties of solubility, water-binding capacity, emulsifying capacity, foaming capacity and stability. The proteins obtained in this way are therefore not very suitable for use in foods for human consumption because, as already explained, they have hardly any functionalities (solubility, foaming, gelling, etc.) and also have a strong off-flavor. They have a large particle size, which results in an unpleasant, sand-like mouthfeel, a coarse granular structure of the proteins and limited film-forming ability.

In order to reduce the above-mentioned disadvantages of obtaining potato proteins by coagulation, further methods have been developed to improve the product quality of the coagulated potato proteins.

International Patent Application Publication WO 2017/142406 describes an improvement in the protein properties (lower hardness, smaller particles, lower density, lower off-flavor) if the coagulated potato protein is washed to a conductivity of the wash water of <1 mS/cm before drying. Likewise, an improvement of the protein properties solubility and water-binding capacity can be achieved by physically reducing the particle size of the protein, as described in WO 2016/133448, or by extraction steps with low molecular weight alcohols such as ethanol or propanol-water mixtures, as set out in WO 2020/171708. In the last example, the precipitation of proteins using organic solvents described above is used. Despite the sensory improvements of the coagulated potato proteins that can be achieved in this way and the resulting application in food, the severe loss of functionality of the proteins caused by denaturation remains irreversible.

The development of methods for the large-scale isolation of potato proteins without denaturation is therefore of great interest. International Patent Application Publication WO 2014/011042 describes the fractionation of proteins from potato amniotic fluids into a high molecular weight fraction and a low molecular weight fraction using a functionalized carrier material utilizing adsorption and desorption steps of the individual fractions. WO 2008/069650 describes the isolation of proteins and the protease inhibitor from potato amniotic fluid using expanded-bed chromatography, wherein a separation into two protein fractions takes place. Pure native potato proteins with high functionality can be obtained, but no total fraction. Chromatographic methods are associated with high process costs, difficult up-scaling, and high technical and energy expenditure, which is why methods without these steps are of great interest. International Patent Application Publication WO 2018/183770 describes the isolation of a dispersible potato protein powder for use in food, feed and beverages with a protein content of 30-91%, a content of α-glycoalkaloids <300 ppm, an ash content of 1-20% and a particle size of 10-100 μm. It includes the following steps:
a. Microfiltration of potato amniotic fluid
b. Ultrafiltration
c. Microparticulation, optionally
d. Spray drying
e. Glycoalkaloid removal before or after ultrafiltration and optional reverse osmosis

In addition, International Patent Application Publication WO2020/242299A1 describes the preparation of a potato protein from peeled potatoes, wherein the protein isolation can also include the step of diafiltration against a salt solution with a conductivity of 5-20 mS/cm in addition to isoelectric precipitation, coagulation, microfiltration and ultrafiltration. In this document, explicit reference is made to the unavoidable peeling of the tubers before processing and this is claimed. Peeling is intended to produce a cleaner protein with a reduced microbial load and lower glycoalkaloid content, which should mean that fewer purification steps are required. In addition, the protein obtained in this way has a different protein composition than that of peeled tubers. While the protein obtained from peeled tubers has higher levels of tyrosine, proline, arginine, glutamine, glutamate, asparagine and aspartate, many essential amino acids are contained more in the skin of the tuber, which are lost during the peeling process. This mainly concerns threonine, leucine, isoleucine, methionine and phenylalanine. In addition, as mentioned above, many minerals are enriched in the skin.

The fact that the extraction of potato proteins from potato amniotic fluid is basically possible by ultrafiltration and diafiltration is already known to the skilled person from the above-mentioned textbook “Advances in Potato Chemistry and Technology”, see above (see also, for example, International Patent Application Publication WO 97/42834; H. J. Zwingenberg, A. J. N. Kemperman, M. E. Boerrigter, M. Lotz, J. F. Dijksterhuis, P. E. Poulsen, G.-H. Koops, Desalination 2002, 144, 331-334; B. J. Oosten, The Starch 1976, 4, 135-137; G. Eriksson, B. Sivik, Potato Res. 1976, 19, 279-287; I. Wojnowska, S. Poznanski, W. Bednarski, J. Food. Sci. 1981, 47, 167-172).

The principle of removing flavorings and contaminants from protein-containing food products using adsorption techniques is also known in principle. For example, International Patent Application Publication WO 2008/069651 describes the adsorption of glycoalkaloids on activated carbon itself or on activated carbon coated with a gel-like agent. German Patent Application Publication DE 29 47 207 A1 from 1979 already describes the removal of undesirable flavorings from a potato albumin fraction from potato amniotic fluid using activated carbon treatment.

Potato patatin, which is found in large quantities in potato protein, can break down fatty acids from triglycerides. The selectivity of this reaction depends strongly on the chain length and the structure of the fatty acids, in particular from P. Pinsirodom, K. L. Parkin, J. Am. Oil Chem. Soc. 1999, 76, 1119-1125 and C. Anderson, P. Pinsirodom, K. L. Parkin, J. Food. Biochem. 2002, 26, 63-74. The basic interaction of lipases with triglycerides and the dependence of their selectivity on the chain length of the fatty acids is a principle that has been known for a long time (see textbooks such as “Advances in Potato Chemistry and Technology”, p. 78, 2^{nd} paragraph), as can also be seen from EP 232933A1 or Römpp, keyword “Lipases” 11^{th} edition.

In order to avoid the formation of off-flavor notes due to this lipase activity and the associated cleavage of fatty acids, patatin is only used in the literature with longer-chain fats (e.g., U.S. Patent Application Publication 2017/0196243), which it cannot cleave. The cleavage of triglycerides, on the other hand, can also be used to deliberately create a flavor note by specifically using fats with shorter chain lengths, as shown in U.S. Patent Application Publication 2009/053191 and by Spelbrink et al. (R. E. J. Spelbrink, H. Lensing, M. R. Egmond, M. L. F. Giuseppin Appl. Biochem. Biotechnol. 2015, 176, 231-243). Preferred oils in which patatin does not break down triglycerides and thus does not cause any changes in taste can be found in the publication: “Fatty Acids Composition of Vegetable Oils and Its Contribution to Dietary Energy Intake and Dependance of Cardiovascular Mortality on Dietary Intake of Fatty Acids” (J. Orsavova, et al., Int. J. Mol. Sci. 2015, 16, 12871-12890), to which reference is made in full. The fact that patatin has this lipase activity was first described by Galliard in 1971 (T. Galliard, Biochem. J. 1971, 121, 379-390). The Wikipedia article from 2008 on patatin (https://en.wikipedia.org/w/index.php?title=Patatin&oldid=193784497) also describes the enzyme activity of patatin as a lipase with the ability to break down fatty acids and refers to the review article “Tuber Storage Proteins” published in 2003 by P. R. Shewry (P. R. Shewry, Ann. Bot. 2003, 91, 755-769), which provides a comprehensive overview of the lipase activity, properties and behavior of patatin. Therefore, when using total potato protein fractions in foods, special consideration must be given to lipase activity by exercising caution when working with fats and, as mentioned above, some fats are more suitable if the formation of off-notes by free fatty acids is undesirable. Inactivation of the enzyme activity, e.g., by heating to the denaturation temperature of the heat-sensitive lipase, can also be helpful.

The known methods for preparing a highly functional potato protein could therefore be improved in various respects.

### SUMMARY OF THE INVENTION

The object of the invention is to obtain a native, functional potato protein of natural structure from potato amniotic fluid in a simple manner on an industrial scale.

The problem is solved by a process as described herein and a potato protein according to the present invention. According to the invention, this can be obtained with a high yield by the process using the key technologies of membrane filtration and adsorbent treatment. The fact that unpeeled potatoes are processed means that a more complete amino acid spectrum is obtained in the product, a time-consuming method step (peeling) is avoided and less waste is produced.

A typical potato protein according to the invention with a molecular weight of 10-120 kDa (according to SDS-Page primary structure) is obtainable by:
a) optionally providing chopped, cleaned potatoes under oxidation protection;
b) mechanical separating the solids from the chopped potatoes to produce potato amniotic fluid and solids;
c) adjusting the pH value of the potato amniotic fluid to a pH value between 6 and 9;
d) separating insoluble components and suspended solids by centrifugal separation;
e) ultrafiltration of the pH-adjusted potato amniotic fluid;

Diafiltration of the ultrafiltration retentate to reduce the electrical conductivity of the retentate solution by 50-90%, preferably 65-90%, particularly preferably 75-85% while retaining the potato protein solution;
a) optionally, drying the potato protein solution, and
b) optionally, sterilizing.

The liquid potato protein ultrafiltration retentate can be converted into dried potato protein by gentle drying. Suitable methods include spray drying, lyophilization, vacuum drying and the like. However, it can also be added to other substances in liquid form.

The highly functional potato protein product obtained economically on an industrial scale is of high quality and has advantageous functional properties for food applications (e.g. gelling, emulsion formation and foaming). However, it can also be used as an adhesive for special applications—for pharmaceutical carrier materials, e.g. cellulose and its derivatives. In contrast to other vegetable proteins, the potato protein according to the invention also offers the ability for hot gelling with high viscosity development. The gel's water-binding capacity results in better mouthfeel and a softer texture.

The taste of the protein was also greatly improved by this treatment.

In one embodiment, the invention relates to a dried native, functional potato protein with a molecular weight of 10-120 kDa (according to SDS-Page primary structure), producible from potato amniotic fluid, obtained by the mechanical solid/liquid separation of chopped, purified potato parts, characterized by:
a) protein content of at least 84% by weight absolute dry
b) humidity content of max. 10% by weight
c) foam volume of 1400-2000 mL
d) Foam stability of at least 90%
e) product solubility/protein solubility in tap water of 75-100%.
f) Maximum gel strength of at least 1000 g
g) ash content of max. 3% by weight.

In one embodiment, the protein according to the invention has a molecular weight of 10-120 kDa (according to SDS-Page primary structure). It has three main fractions, the first main fraction having a molecular weight of 10 to 20 kDa, the second main fraction having a molecular weight of between 25 and 40 kDa and the third main fraction having a molecular weight of about 66 kDa according to SDS-Page primary structure. According to HPLC chromatography, at least 70% of the proteins have a molecular weight of 3-182 kDa. The different detected molecule size ranges are due to the different analytical methods. While the mass of the protein in its primary structure is determined in the SDS-Page, the volume of the proteins present in their quaternary structure is determined in HPLC chromatography.

In one embodiment of the invention, the protein according to the invention is obtainable by:
a) providing chopped, cleaned potato parts;
b) mechanical separating the solids from the chopped potatoes to produce potato amniotic fluid and solids;
c) adjusting the pH value of the potato amniotic fluid to a pH value between 6 and 9;
d) separating insoluble components and suspended solids by centrifugal separation;
e) ultrafiltration of the pH-adjusted potato amniotic fluid;
d) diafiltration of the ultrafiltration retentate to reduce the electrical conductivity of the retentate solution by 50-90%, preferably 65-90%, particularly preferably 75-85%.
   In one embodiment of the invention, the dried protein according to the invention is obtainable by adjusting the pH value after the mechanical solid/liquid separation to a pH value of 6-9, preferably 6.5-8.2, particularly preferably 6.9-7.5.

In one embodiment of the invention, the dried protein according to the invention is obtained by drying by at least one of the method steps of spray drying, lyophilization, vacuum drying, freeze drying.

In one embodiment of the invention, the protein according to the invention is obtainable in that the ultrafiltration membrane is a plastic or ceramic membrane with a cut-off of 3-150 kDa, preferably 5-120 kDa, particularly preferably 10-100 kDa.

In one embodiment of the invention, the protein according to the invention is characterized in that the ultrafiltration retentate is treated with adsorbents selected from activated carbon, bentonites, polymeric adsorbents or derivatives of the adsorbents. Typical polymeric adsorbents are polystyrene/divinylbenzene resins, phenol formaldehyde resins, derivatives of the adsorbents and combinations thereof, which decolorize and remove off-flavors. In a preferred embodiment, the polymeric adsorbent used is a polystyrene/divinylbenzene resin. The resins Amberlite XAD 761, Amberlite FPX 68, Diaion HP 20, Sepabeads SP 70, Purosorb PAD 550 polymer adsorbents, activated carbon, cellulose-based adsorbents, gelatine, cellulose-based adsorbents, tannins, are also preferably used for the reduction of off-flavors-polystyrene/divinylbenzene resins, polyphenol-formaldehyde resins and combinations thereof can also be used.

In one embodiment of the invention, a protein according to the invention can be produced in that the ultrafiltration retentate is gently sterilized, for example by pasteurization with microwaves or HTST pasteurization (High Temperature Short Time), PEF sterilization (Pulsed Electric Fields), HPP pasteurization (High Pressure Pasteurization).

In one embodiment of the invention, the protein according to the invention is obtainable by:
providing the chopped, cleaned potato parts in the form of potato mash, mechanical separation of the potato mash into solid and liquid phase (potato amniotic fluid);
adjusting the pH value to 6-9, preferably 6.5-8.2, particularly preferably 6.9-7.5.
ultrafiltration of the liquid phase with membranes with a cut-off of 3-150 kDa, preferably 5-120 kDa, particularly preferably 10-100 kDa;
diafiltration of the ultrafiltration retentate up to a reduction in conductivity of 50-90%, preferably 65-90% and particularly preferably 75-85%.
removing antinutritive substances using adsorption techniques;
optionally, sterilizing and
optionally, drying the protein solution.

In one embodiment of the invention, the protein according to the invention is a component of a food or food additive, a dietary food or food additive for human or animal consumption.

In one embodiment of the invention, the protein according to the invention is a component of foods or food supplements, dietary foods or food additives for human or animal consumption, both as a solution and as a solid.

In one embodiment of the invention, the protein according to the invention is a component of an adhesive.

In one embodiment of the invention, the protein according to the invention is a component of a mixture with other vegetable proteins, preferably legume proteins, such as pea protein, bean protein, field bean protein, lentil protein or soy protein, lupin protein or mung bean protein or mixtures thereof.

### Preparation of Native Functional Potato Protein:

The potato amniotic fluid from washed potatoes with skin is obtained after solid/liquid separation of chopped, cleaned potato parts, for example as a side stream of potato starch preparation. The first step is to remove sand, stones and plant residues from the potato raw material and to chop it, e.g., using a grater, usually with the addition of a reducing or antioxidant agent, such as sodium hydrogen sulphite, or in an inert gas atmosphere. Prior peeling of the potato is undesirable for carrying out the method according to the invention, which makes it an advantageous further development in contrast to prior art methods in which peeling of the potatoes is explicitly described. By dispensing with peeling, the economy of the method is improved and a nutritionally more favorable amino acid spectrum is obtained, as previously explained. With conventional peeling, a peeling loss of 10% by weight can be assumed, which means that with an initial quantity of 1000 tons of potatoes, a loss of 100 tons of raw material is generated through peeling. Added to this are savings in energy and electricity costs, as well as material costs that would be incurred by changing blades or replacing the roller peelers in a mechanical peeling process. With steam peeling, the energy costs must also be taken into account, as well as the costs of electricity, water, and heating. In addition, the potato is exposed to elevated temperatures during steam peeling, which can lead to denaturation of the protein and, due to gelatinization of the starch, make it difficult or impossible to isolate the protein.

Centrifugation is used to separate the product into a solid phase consisting of starch and fibers, and potato amniotic fluid water as a liquid phase with water-soluble components such as proteins, sugars, amino acids, organic acids, and salts, etc. These steps are common in this or a similar way for the extraction of starch from potatoes (see, for example: J. BeMiller, R. Whistler, Starch: Chemistry and Technology, 3^{rd} edition, Academic Press, pp. 522-555).

For a possible preparation method of the native functional potato protein from potato amniotic fluid, its pH value is first adjusted to 6-9, preferably 6.5-8.2, particularly preferably 6.9-7.5, with the aid of a food-safe aqueous lye, for example sodium hydroxide solution, potassium hydroxide solution or calcium hydroxide. Insoluble components and suspended solids are then separated by centrifugal separation. Through these first process steps, the potato amniotic fluid loses its turbidity and becomes a clear protein solution. By ultrafiltration of the separated clear protein solution, a concentration of the proteins in the liquid ultrafiltration retentate, the protein solution, is achieved. In one embodiment, the concentration factor is about 10, wherein a higher or lower protein concentration is also possible, depending on conditions such as energy costs.

Ceramic and plastic diaphragms are preferred. For example, a plastic membrane with a cut-off of 3-150 kDa, preferably 5-120 kDa, particularly preferably 10-100 kDa, is suitable for this purpose. Membrane filtration is carried out at low pressures of 1-3 bar. Excessive pressure is avoided, as this mechanical stress has a negative effect on the functionality of the proteins.

Before or after ultrafiltration, undesirable flavorings and antinutritive substances, including glycoalkaloids, can be removed from the protein solution using adsorption techniques. Activated carbon or bentonite adsorption is particularly suitable, but other adsorbents as mentioned above can also be used.

The ultrafiltration retentate is diafiltered until the electrical conductivity is reduced by 50-90%, preferably 65-90%, particularly preferably 75-85%. The diafiltration can be carried out with fully deionized water, distilled water, process water or tap or process water, wherein the water used can have a conductivity of 5 μS/cm-5000 μS/cm, preferably 10-50 μS/cm or even 5-50 μS/cm.

The resulting protein solution can then optionally be sterilized using gentle methods. Pasteurization with microwaves, HTST pasteurization (High Temperature Short Time), PEF sterilization (Pulsed Electric Fields) or HPP pasteurization (High Pressure Pasteurization) are suitable for this. Finally, the protein solution can be dried by spray drying or other gentle drying methods, e.g. lyophilization or vacuum drying. However, further use of the protein solution is also conceivable.

The potato protein according to the invention with a molecular weight of 10 to 120 kDa (according to SDS-Page primary structure) is characterized by its good emulsifying and foaming properties. It is also hot-swelling, which enables better processing and easier protein enrichment. The low viscosity in the cold state also leads to good processability, whereas many other commercially available proteins already have a significantly higher viscosity here. The superior gel-forming properties when heated are similar to animal proteins and make the potato protein according to the invention highly interesting for use in meat substitute products, among other things.

In the following, the invention is explained by means of the exemplary embodiments as well as the drawings, to which it is by no means limited.

### BRIEF DESCRIPTION OF THE DRAWINGS

FIG. 1 shows a method diagram of preparation example 1.

FIG. 2 is a HPLC chromatogram of the potato protein of the invention and a protein standard.

FIG. 3 is a HPLC chromatogram of the potato protein of the invention and Solanic 300®.

FIG. 4 is a HPLC chromatogram of the potato protein of the invention and Solanic 200®.

FIG. 5 shows a SDS-Page gel of the potato protein of the invention (lane 1=marker; 2 and 3: batches of the potato protein of the invention)

FIG. 6 shows DSC diagrams for thermally treated and thermally untreated potato proteins.

FIG. 7 shows a viscosity measurement after Anton Paar of the potato protein according to the invention in comparison with a sample treated with calcium chloride.

FIG. 8 shows Gel strength measurements of potato protein according to the invention in comparison with a sample treated with calcium chloride.

FIG. 9 is a diagram show a method in accordance with the invention.

### DETAILED DESCRIPTION OF THE INVENTION

### Examples of the Preparation Method

### Example 1—Preparation of Highly Water-Soluble, Functional Potato Protein

Potatoes were cleaned and finely chopped to produce an embodiment of highly water-soluble potato protein. The suspension was subjected to gravity separation (centrifugation) and the supernatant was used as protein-rich amniotic fluid for protein extraction. The protein-containing, cloudy solution was adjusted to a pH value of 7.0 to 8.0 with the aid of aqueous sodium hydroxide solution and centrifuged again, wherein fine suspended particles were removed from the solution. The purified protein-containing clear solution was ultrafiltrated using a 100 kDa polyvinylidene fluoride membrane at a transmembrane pressure of 2.0 bar and a differential pressure of 1.0 bar. The protein according to the invention remained in the ultrafiltration retentate, while salts, sugars, and amino acids remained in the ultrafiltration permeate. The ultrafiltration retentate was then diafiltered with demineralized water until the electrical conductivity was reduced by 81%. Subsequently, the antinutritive components were removed and decolorized using activated carbon adsorption. The potato protein obtained after spray drying according to the invention had the following properties:

| | |
|---|---|
| Moisture [% by weight] | 5.6 |
| Protein content [% by weight absolute dry] | 87.5 |
| Product solubility [% by weight] | 82.4 |
| Ash [% by weight] | 1.69 |
| Foam activity [mL] | 1500 |
| Foam stability [%] | 100 |

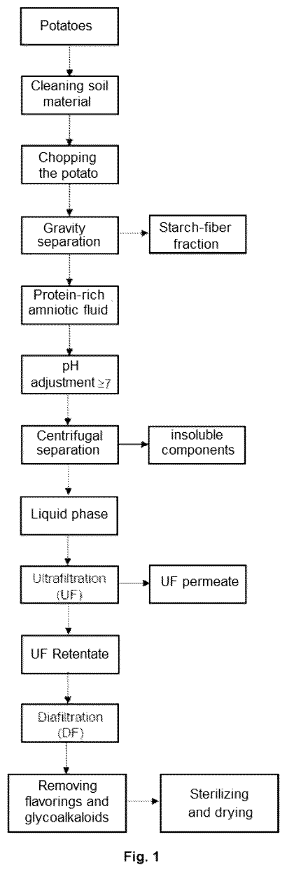

The method steps of Example 1 are illustrated in FIG. 1. Example 1 represents an exemplary embodiment of the invention, but is by no means limited thereto.

### Example 2—Preparation of a Functional Potato Protein Solution

Potatoes were cleaned and finely chopped to produce an embodiment of highly water-soluble potato protein. The suspension was subjected to gravity separation (centrifugation), and the supernatant was used as protein-rich amniotic fluid for protein extraction. The protein-containing, cloudy solution was adjusted to a pH value of 7.0 to 8.0 with the aid of aqueous sodium hydroxide solution and centrifuged again, wherein fine suspended particles were removed from the solution. The purified protein-containing clear solution was ultrafiltrated using a 100 kDa polyvinylidene fluoride membrane at a transmembrane pressure of 2.0 bar and a differential pressure of 1.0 bar. The protein according to the invention remained in the ultrafiltration retentate, while salts, sugars and amino acids remained in the ultrafiltration permeate. The ultrafiltration retentate was then diafiltered with demineralized water until the electrical conductivity was reduced by 81%. Subsequently, the antinutritive components were removed and decolorized using activated carbon adsorption. The potato protein solution obtained had:

| | |
|---|---|
| Moisture [% by weight] | 81.1 |
| Protein content [% by weight absolute dry] | 86.7 |
| Product solubility [% by weight] | 91.2 |
| Ash [% by weight] | 2.09 |

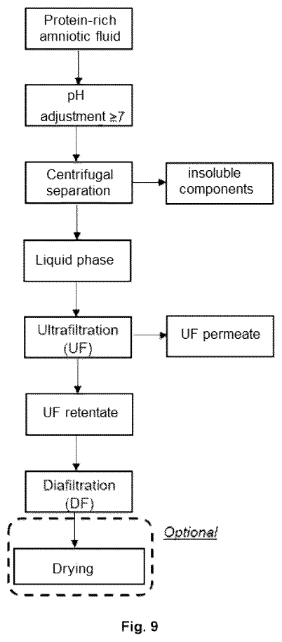

The method steps of Example 2 are illustrated in FIG. 9. Example 2 represents an exemplary embodiment of the invention but is by no means limited thereto.

Further examples of applications are given below which show possible uses of the water-soluble protein according to the invention; further applications are obvious to the person skilled in the art.

### Example 3: HPLC

The potato protein according to Example 1 was analyzed using an HPLC from Knauer. An HPLC Xbridge BEH SEC 200A, 3.5 from Waters was used as the column and eluted with an aqueous solution of 0.02 M Na₂HPO₄/NaH₂PO₄ at pH 7. As the standards, the followings were used from Sigma-Aldrich:
670 kDa—Thyroglobulin
150 kDa—Gamma-Globulin
44.3 kDa—Ovalbumin
13.74 kDa—Ribonuclease A

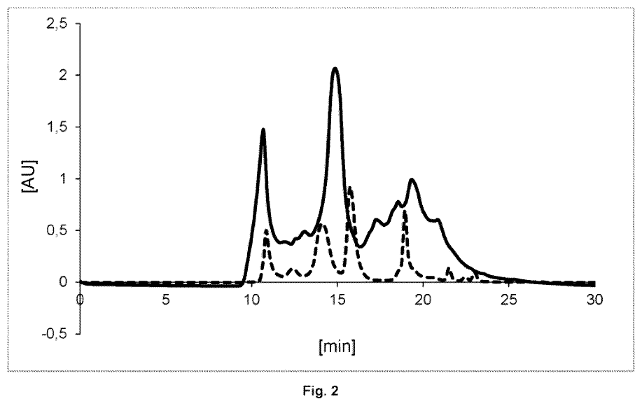

UV absorption at 214 nm was used for detection. The measured HPLC chromatogram is shown in FIG. 2, wherein the time in minutes is plotted against the absorbance in absorbance units. FIG. 2 shows the protein standard (broken lines) with relatively sharp peaks at 10.84 min for thyroglobulin; 14.12 min for gamma globulin; 15.74 min for ovalbumin, and 18.93 min for ribonuclease. The chromatogram of the protein of the invention (solid line) was superimposed on that of the standards. Different protein fractions are clearly recognizable, with two main components occurring at retention times of about 10 and 15 minutes, and a mixture of different proteins in the range of 17-20 minutes.

When evaluating the HPLC chromatograms, it is assumed that the volume corresponds to the peak area of the signals. An evaluation of the volume distribution showed that the relative peak ratios of both the protein standard and the potato protein according to the invention do not change, even at different detector wavelengths. Therefore, a semi-quantitative statement about the volume distribution and a conclusion from the volume distribution to their molar masses is possible. The volume of the proteins in the potato protein according to the invention can therefore be assigned semi-quantitatively to the molar masses: Molar masses and retention times of the potato protein according to the invention:

| Max RT, min | | Start RT, min | | End RT, min | | |
|---|---|---|---|---|---|---|
| (Molar mass, Da) | | (Molar mass, Da) | | (Molar mass, Da) | | Area, % |
| 10.68 | (697.000) | 8.90 | (1.670.000) | 11.67 | (428.000) | 15.8 |
| 11.91 | (381.000) | 11.67 | (428.000) | 12.29 | (315.000) | 2.8 |
| 13.07 | (215.000) | 12.29 | (315.000) | 13.41 | (182.000) | 5.7 |
| 14.85 | (89.700) | 13.41 | (182.000) | 16.32 | (43.500) | 31.7 |
| 17.26 | (27.400) | 16.32 | (43.500) | 17.72 | (21.800) | 7.9 |
| 18.53 | (14.700) | 17.72 | (21.800) | 18.77 | (13.000) | 7.9 |
| 19.33 | (9900) | 18.77 | (13.000) | 20.65 | (5200) | 16.2 |
| 20.84 | (4700) | 20.65 | (5200) | 29.98 | (53) | 11.9 |

The first characteristic signal at retention times of 8.9 to 11.7 min can be assigned to a molar mass of 697 kDa in its maximum at 10.7 min. The main fraction (30% of the total fraction, retention time: 13.41-16.32 min) can be assigned molar masses of 43.5 kDa to 182.0 kDa. In addition, the potato protein according to the invention is composed of a mixture of smaller proteins (retention time: 16.3-30.0 min). Proteins with molecular masses of 5.2 to 13 kDa make up the largest proportion within this low molecular weight range.

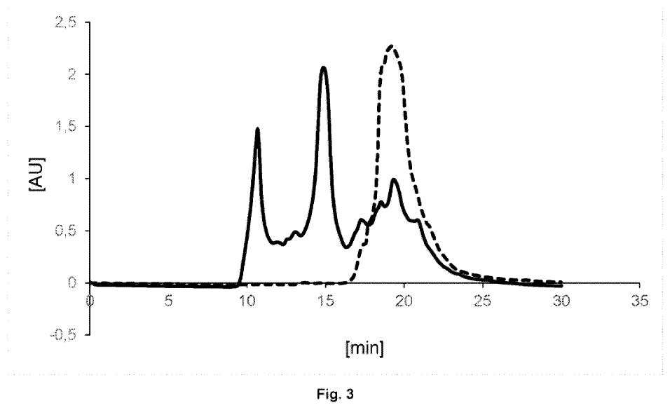

FIG. 3 shows an HPLC chromatogram of the potato protein according to the invention (solid line) and the commercially available potato protein Solanic 300® from AVEBE (broken line), wherein the time in minutes is plotted against the absorbance in absorbance units. A comparison of the two chromatograms clearly shows that Solanic 300® is a single fraction (99%, retention time: 15.5-27.4 min) of a specific molecular weight range.

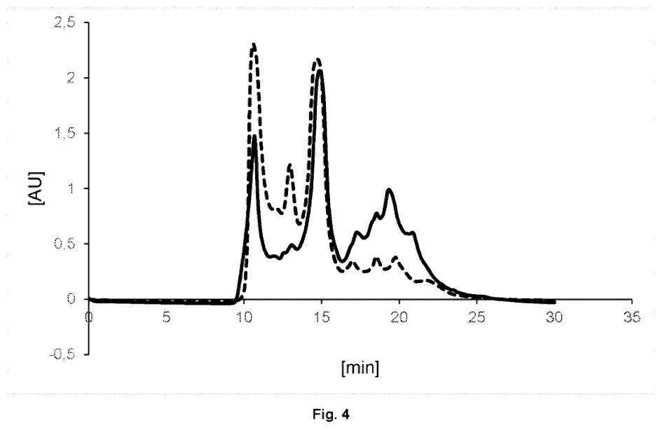

FIG. 4 shows an HPLC chromatogram of the potato protein according to the invention (solid line) and the commercially available product Solanic 200® from AVEBE (broken line), wherein the time in minutes is plotted against the absorbance in absorbance units. While the protease inhibitor fraction (Pl fraction) is missing in Solanic 200®, it is still present in the potato protein according to the invention, which represents a water-soluble total fraction of the potato protein.

Compared to the Solanic® products, the potato protein according to the invention represents a water-soluble total fraction of the potato protein. This makes it the only commercially and economically producible highly functional total potato protein fraction. To date, only functional single fractions or coagulated total protein fractions with significantly limited functionality have been available. Total potato protein is defined here as the total fraction of water-soluble proteins. Other commercial potato proteins, such as KMC's Protafy 130®, could not be analyzed by HPLC due to insufficient solubility.

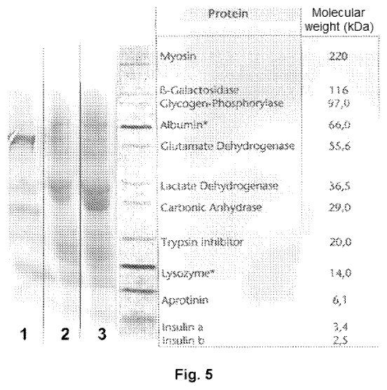

a) The potato protein according to the invention was also analyzed using SDS-Page gel chromatography—see FIG. 5. The selectivity of the method is clearly recognizable, whereby proteins with a molecular weight >120 kDa are not present in the product. Here, as in the HPLC chromatogram, three most intense areas are recognizable and can be assigned molecular weights of 10-20 kDa, 25-40 kDa and about 66 kDa according to the SDS-Page.

b) However, the two analysis methods are not comparable in terms of the molecular weights determined, as the proteins are denatured differently in the measurement methods. Nevertheless, both methods show that three protein mixtures are the main components of the potato protein according to the invention. According to SDS-Page, this corresponds to ranges of 10-20 kDa, 25-40 kDa and a mixture around 66 kDa, while the potato protein according to the invention is composed of a protein mixture of 428-1627 kDa, the main fraction of 43-182 kDa and a mixture of smaller proteins with a main fraction at 5-13 kDa according to HPLC chromatography.

c) The discrepancy in the detected molecule sizes is due to the different analytical methods: While the mass of the proteins in their primary structure is determined in the SDS-Page, the volume of the proteins is determined in HPLC chromatography. Here the proteins are still in their quaternary structure. The primary structure of the protein corresponds to its amino acid sequence in an elongated form, whereas in the quaternary structure the protein is present in a spatial structure as a protein complex-thus there is a difference in the binding ratios present. For this reason, the specified molecule sizes initially appear very different.

### d) Example 4: Differential Scanning Calorimetry (DSC) Analysis

e) Denaturation of a protein means that the folding state of the protein is changed to a protein structure of a lower order. The unfolding of a protein is an energetic balance of different interactions between protein groups and the surrounding medium, so that the matrix in which the protein is dissolved influences the amount of energy absorbed during denaturation.

f) These folding and unfolding reactions are associated with heat effects that can be investigated using differential scanning calorimetry (DSC) analysis. In DSC analysis, the heat capacity of a protein in aqueous solution is measured as a function of temperature, wherein the area under the heat capacity curve corresponds to the enthalpy of denaturation. A higher enthalpy, i.e. more heat required to destroy the ordered protein structure, indicates a higher degree of folding of the protein. The peak temperature of the heat capacity profile (T_{dPeak}) corresponds to the transition temperature.

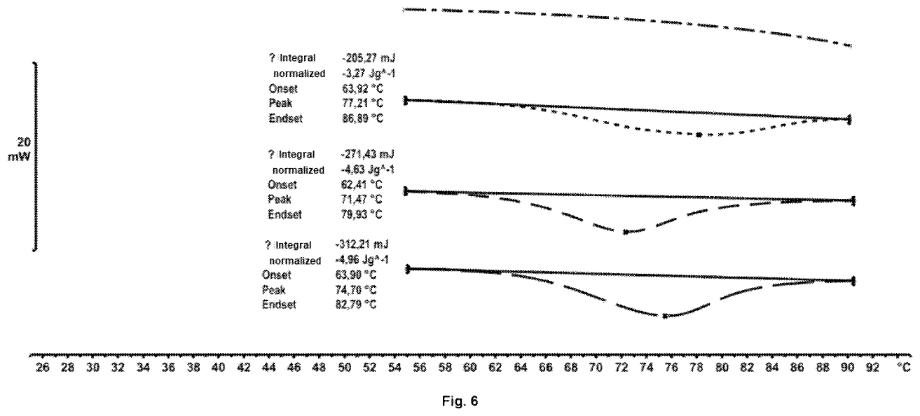

g) For the measurements, approximately 60 mg of a 50% commercial solution of the protein to be tested in demineralized water was weighed into a 100 μL aluminium crucible. The measurement was carried out on a DSC3+ Star System from Mettler Toledo® in a nitrogen atmosphere, wherein a temperature range of 25-95° C. with a heating rate of 10° C./min was investigated. FIG. 6 shows that the animal feed potato protein (dot-dashed line, first curve), which is a protein coagulated by increasing the temperature and adjusting the pH value, does not absorb any heat. This is due to the complete denaturation of the protein. In contrast, the potato protein according to the invention (dotted line, second curve) shows heat absorption from a temperature of approx. 64° C., which corresponds to the start of denaturation (Tₒₙₛₑₜ). The heat absorption ends at approx. 87° C., resulting in a denaturation peak temperature (T_{dPeak}) of approx. 77° C. The commercially available potato proteins Solanic 300® (short dashed line, third curve) and Solanic 200® (long dashed line, fourth curve) were then examined for comparison. As described above, Solanic 300® is a mixture of different proteins of low molecular weight, which can be summarized as a protease inhibitor fraction (Pl fraction). Solanic 200®. on the other hand, is a refined patatin in which the protease inhibitors (Pl fraction) are almost completely absent. This is reflected in the DSC measurement.

h) Solanic 200® starts to absorb heat from a temperature of 62° C., wherein the heat absorption ends at 79° C. (T_{dPeak}=72° C.). Solanic 300®, on the other hand, absorbs heat in a similar way to the potato protein according to the invention from approx. 64° C., although this ends at approx. 83° C.

i) The temperature profiles generally show that all proteins are clearly different products. In general, larger proteins are denatured more easily, i.e., at lower temperatures, than smaller proteins, which can no longer be denatured once they reach a certain size. Solanic 300® therefore has a longer heat absorption period, as it contains the Pl fraction, unlike Solanic 200®. Based on the significantly longer heat absorption of the potato protein according to the invention, it is clear that it has a different protein fraction in which small proteins are still contained.

### Example 5: Analytical Characterization

Dried potato protein according to the invention, producible according to example 1, was examined.

### Analytical Methods:

### Humidity Determination:

Device: Surface dryer (drying temperature 105° C. Step 2)

### Protein Content:

Nitrogen determination according to Kjeldahl (Nx6.25), DIN EN ISO 3188

### Product Solubility:

Weighing: 40 g tap water+0.5 g product
Stirring time: 1 h
Fill up to 50 mL in the volumetric flask
Centrifuge for 30 min at 2770×g
j) Filter via Whatmann filter (No. 1) (paper filter with 11 micrometer pore size)
k) Weigh 20-25 g of filtrate into a glass dish
l) Dry for 24 hours in a drying cabinet at 100° C.

$$\begin{matrix}m ) & Calculation : 0.5\end{matrix} \cdot \frac{Dry substance filtrate [ \% ]}{0.5 g - Humidity [ \% ]} \cdot 10000$$

### n) Protein Solubility:

o) See product solubility

p) Nitrogen was determined from the filtrate according to Kjeldahl (Nx6.25), DIN EN ISO

q) 3188
r) Weighing: approx. 6 g filtrate

s) The solubilities of the dried potato protein according to the invention are strongly dependent on the salt content of the water used for the analysis.

### t) Ash:

u) Weighing: approx. 1 g product
v) Microwave oven: MAS 7000
w) Ashing temperature: 550° C.
x) Ashing time: 60 minutes

### Foam Activity and Stability:

y) Dissolve 5 g product in 95 g demineralized H₂O
z) Whip for 15 minutes on speed 3 (Hobart 50-N)
aa) Determine foam volume=Foam activity in mL
bb) Determine foam volume after 60 min. standing time=Foam stability in %

### Emulsion Capacity:

cc) Weighing: 80 g demin. H₂O+10 g product
dd) Pour 250 mL sunflower oil into a dropping funnel
ee) Stir with Ultra Turrax at 20000 rpm, maintain a temperature of 20° C.
ff) Add sunflower oil continuously until phase inversion (until emulsion
   gg) suddenly becomes thinner)
hh) Determine the volume of unconsumed sunflower oil

ii) 250 mL−remaining volume=consumption (sunflower oil)
jj) Result: 10 g product: 80 g demin. H₂O: Consumption/10
   kk) Viscosity measurement using Brookfield HAT, spindle 4, 20 rpm

### Viscosity:

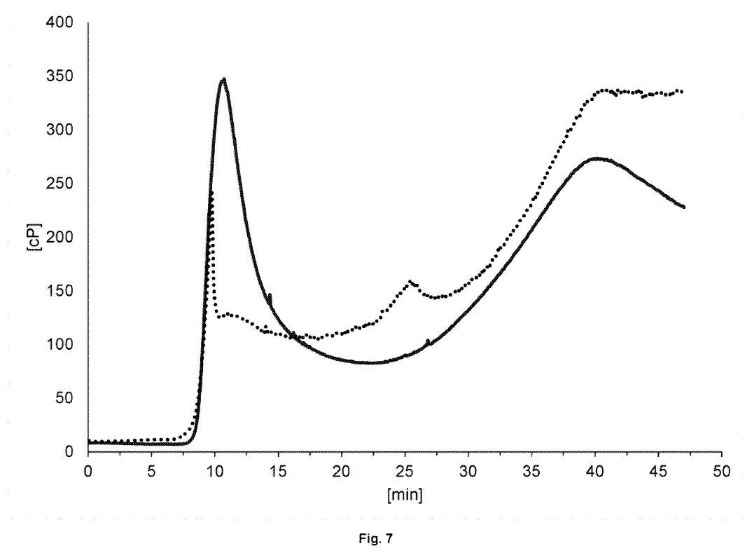

ll) FIG. 7 shows tests on the viscosity of the dried potato protein according to the invention (solid line) in comparison with a sample treated with calcium chloride (dotted line), wherein the time in minutes is plotted against the viscosity in cP. For the preparation of the calcium chloride-treated sample, 0.5% by weight calcium chloride based on the mass of the potato amniotic fluid was added as an aqueous solution (CaCl₂):water=1:3) after mechanical separation of the solids and before adjustment of the pH value and centrifugal separation, whereupon all further process steps were carried out as described above (see FIG. 1). The viscosity profiles were recorded as follows: A 15% solution of the product in demineralized water was prepared. In the Anton Paar Physica MCR 301 (standard insert, stirrer ST24-2D, 60 rpm), 35 mL of the solution was measured according to the temperature profile (start: 25° C., heating 6.5° C./min, holding at 90° C. for 12 min, 25 cooling at 4.3° C./min, holding at 25° C. for 10 min). FIG. 7 clearly shows that the CaCl₂) treatment of the protein has a significant influence on the viscosity of the protein. The potato protein according to the invention has a viscosity of about 230 cP when the temperature of 90° C. is reached, which increases to about 335 cP at the end of the cooling period and thus forms a solid gel. The addition of CaCl₂) even increases the viscosity to around 350 cP when the maximum temperature of 90° C. is reached, but the gel formed is not stable. During cooling, the gel strength decreases continuously, so that at the end of the measurement it is only around 225 cP, which is significantly lower than that of the potato protein according to the invention.

### Gel Strength:

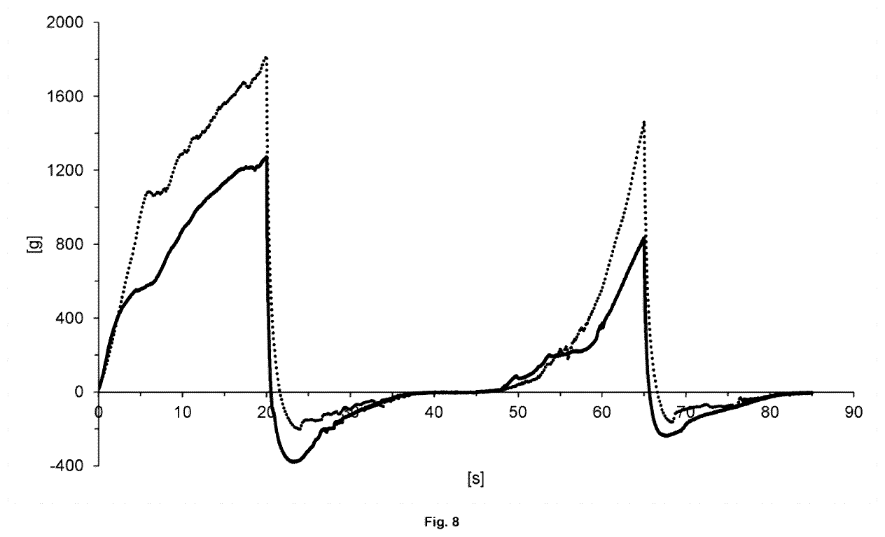

mm) FIG. 8 shows experiments on the gel formation behavior of the proteins, where the time in seconds is plotted against the force in grams. The potato protein according to the invention (solid curve) was compared with a sample of potato protein treated with CaCl₂) (dotted line) in order to illustrate the influence of the salt on the protein. Again, for the preparation of the calcium chloride-treated sample, 0.5% by weight calcium chloride based on the mass of the potato amniotic fluid was added as an aqueous solution (CaCl₂):water=1:3) after the mechanical separation of the solids and before adjustment of the pH value and centrifugal separation, whereupon all further process steps were carried out as described above (see FIG. 1). The gel strength was examined by means of texture analysis with the TA XT plus Texture Analyzer using the stamp (SMS P 05) (distance: 20 mm, forward, test and return speed: 1.0 mm/sec; release force: 20 g) at room temperature.
nn) Preparing 2 samples: Provide protein (6 g or 12 g product) and add demin. H₂O (34 g or 68 g), stirring until the protein is dissolved
oo) Pour 30 mL of the sample solution into an Anton Paar rheometer with metal cylinder with hole (H-CC27-D)
pp) Boiling the sample solution: starting temperature of 25° C., heating phase: heating to 90° C. at a heating rate of 6.5° C./min, holding time at 90° C. for 15 min, cooling phase: cooling to 25° C. at a cooling rate of 4.0° C., holding time at 25° C. for 10 min
qq) Store boiled sample solution for 24 h at room temperature
   rr) Texture Analysis with the TA XT Plus Texture Analyzer

ss) In the measurement method, a plunger is slowly pressed into the prepared sample solution, which corresponds to the first peak. When moving the plunger into the gel, a force must be applied until the plunger has completely penetrated the gel. The negative force then corresponds to the retraction of the plunger and the elastic tightening of the gel. The process is then repeated, and the plunger penetrates the gel a second time. The maximum force is usually lower in the second process, since the gel strength is still impaired by the first process. The more similar the peaks are, the greater the elasticity of the gel.

tt) You can clearly see that different forces are required to penetrate the gel. While the potato protein according to the invention requires a maximum force of 1265 g to be applied at the first peak, this is reduced by 34% to 836 g when the stamp is pushed through the second time. Treatment with calcium chloride has a strong influence on the gel strength of the protein. The measurement illustrates the strong dependence of the protein on external factors, as already explained in the state of the art. The treated sample exhibits a maximum force of 1815 g at the first peak, which is only reduced by 24% (1468 g) at the second peak. This means that the CaCl₂) treatment significantly increases the gel strength of the potato protein and slightly improves its elasticity.

Chemical Test Results:
Sample no.: 209-2022-00035006
Sample Designation: Empro K

| | | | Measurement |
|---|---|---|---|
| | Method | Unit | value |
| Parameters | | | |
| Total Protein (Dumas) | AOAC 968.06 (mod.), AOAC 992.5 | % | 84.2 |
| | (mod.) | | |
| Total Tryptophan | Rapid, Accurate, Sensitive, and | mg/g | 10.3 |
| | Reproducible HPLC, Agilent | | |
| | Application Note 5990-4547 (2010) | | |
| Amino acid Profile | | | |
| Tyrosine | Agilent Application Note 5990-4547 | mg/g | 48.5 |
| Valine | (2010); Analytical Biochemistry 177, | mg/g | 53.0 |
| Threonine | 318-322. | mg/g | 49.9 |
| Arginine | | mg/g | 43.5 |
| Serine | | mg/g | 46.4 |
| Proline | | mg/g | 43.4 |
| Glutamic acid (E620) | | mg/g | 92.0 |
| Glycine | | mg/g | 42.3 |
| Histidine | | mg/g | 17.8 |
| Isoleucine | | mg/g | 47.0 |
| Leucine | | mg/g | 85.5 |
| Lysine | | mg/g | 66.0 |
| Methionine | | mg/g | 18.7 |
| Phenylalanine | | mg/g | 53.6 |
| Cystine | | mg/g | 14.6 |
| Asparagic acid | | mg/g | 104 |
| Alanine | | mg/g | 42.4 |
| PDCAAS | | | |
| Amino acid index | Federal Register, Vol. 58, No. 3, p. | | 1.11 |
| Protein digestibility | 2194. | | 0.94 |
| Protein digestibility | | | 1.00 |
| (corrected Am) | | | |
| General analytics | | | |
| Total dietary fiber | AOAC 991.43: 1994, mod., CLF SOP | g/100 g | <0.5* |
| | 521-07 (2020 February), Gravimetry: | | |
| Calorific value kJ | According to VO (EU) 1169/2011, CLF | kJ/100 g | 1561 |
| | SOP 521-11 (2020 February), Calculation | | |
| Calorific value kcal | According to VO (EU) 1169/2011, CLF | kcal/100 g | 367 |
| | SOP 521-11, (2020 February), Calculation | | |
| Fat | DIN 10342: 1992 September, mod., CLF SOP | g/100 g | 0.44 ± 0.4 |
| | 521-01, (2020 February), Gravimetry | | |
| Fatty acids | CLF SOP 527-02, GC-FID | % | Not |
| (unsaturated and | | | evaluable |
| saturated) | | | due to low |
| | | | content |
| Carbohydrates | VO (EU) 1169/2011, CLF SOP 521- | g/100 g | 8.2 |
| | 11, (2020 February), Calculation | | |
| Protein (N x 6.25) | § 64 LFGB L 06.00-7: 2014 August, mod., | g/100 g | 82.67 |
| | ASU L17.00-15: 2013 August mod., DIN EN | | |
| | ISO 8968-1: 2014 June, (Modification: | | |
| | Factor = N × 6.5 | | |
| Water (humidity) | ASU L 02.06-E(EG) & 1(EG)- 8(EG) | g/100 g | 6.18 ± 0.618 |
| (102° C.) | Method 2: 1981 January, CLF SOP 521-03 | | |
| | (2020 February), Gravimetry | | |
| Fructose | CLF SOP 522-01 (2020 February), LC-RI | g/100 g | <0.1* |
| Glucose | | g/100 g | <0.1* |
| Sucrose | | g/100 g | <0.1* |
| Maltose | | g/100 g | <0.2* |
| Lactose | | g/100 g | <0.2* |
| Total Mono- and | | g/100 g | <0.7* |
| Disaccharides | | | |
| Ash | ASU L17.00-3, 1982 May, Ber. 2002 December, | g/100 g | 2.54 ± 0.254 |
| | CLF SOP 521-04 (2020 February), Gravimetry | | |
| Sodium (Na) | prDIN EN 16943: 2016 January, mod., CLF | mg/100 g | 118.8 ± 23.8 |
| | SOP 526-03 (2020 February), ICP-OES | | |
| Common salt | calculated from Sodium | mg/100 g | 297.0 |

Result +/− expanded measurement uncertainty (95%, k = 2), sampling not included.
*= The specified value corresponds to the limit of quantification

uu) The above protein analysis of a single batch of protein obtained is only exemplary for the protein according to the invention, which is subject to variations depending on potato growth conditions and storage.

vv) Examples of applications are given below which show possible uses of the potato protein according to the invention. However, its applicability is not limited to these—other applications are obvious to the person skilled in the art.

### Example 6—Chocolate Mousse

| Ingredients | Concentration [%] |
|---|---|
| Potato protein according to the invention | 41.18 |
| (3% solution) | |
| Couverture (dark chocolate) | 58.82 |

ww) Adding the potato protein according to the invention to water while stirring on a stirring plate (3 minutes, approx. 1000 rpm) to produce a 3% solution
xx) Melting the couverture in the Thermomix® by heating to 65° C. at a speed of 4-5
yy) Adding the previously prepared 3% solution of the potato protein according to the invention to the bowl of the Hobart® and beating until stiff (level 3 for 5 minutes)
zz) Slightly cooling the melted chocolate and stirring into the stiff solution of the potato protein according to the invention
aaa) Store the mousse in the fridge for at least 2 hours

bbb) The mousse made with the potato protein according to the invention showed a better structure and was more airy compared to a mousse made with egg white. In addition, the mousse produced with the protein according to the invention showed greater storage stability, as the airy structure was still unchanged after 4 days, whereas the mousse produced with egg white hardened. In addition, the mousse produced with the potato protein according to the invention had a better mouthfeel and melted on the tongue. Another major advantage was that the mousse produced in this way is salmonella-free, whereas the use of fresh eggs can always be associated with the risk of salmonella poisoning. Finally, by using the potato protein according to the invention, a smoother structure with a more attractive chocolate coloring could be achieved compared to egg white.

### Example 7—Yellow Cake

| Ingredients | Concentration [%] |
|---|---|
| Potato protein according to the invention | 11.1 |
| (3% solution in water) | |
| Sugar | 20.0 |
| Butter | 20.0 |
| Flour | 27.8 |
| Milk (3.5% fat) | 20 |
| Baking powder | 1.1 |

ccc) Beat the 3% solution of the potato protein according to the invention until stiff
ddd) Mix the sugar and butter
eee) Add the flour, milk and baking powder to the butter and sugar mixture
fff) Fold in the stiffly whipped solution of potato protein according to the invention with a spoon
ggg) Pour the batter into a loaf pan
hhh) Bake for 50-60 min at 170° C. fan oven

iii) By using the potato protein according to the invention, a fluffy dough was achieved. The potato protein according to the invention is therefore very suitable as a vegan chicken protein substitute.

### Example 8—French Meringue

| Ingredients | Amount [g] |
|---|---|
| Potato protein according to the invention (3% solution) | 100 |
| Sugar | 150 |
| Optional. Xanthan gum | 0.5 |

jjj) Beat the solution of potato protein according to the invention (optionally with the addition of xanthan gum) in the KitchenAid® for 5 minutes until stiff
kkk) Slowly add the sugar while stirring
lll) Continue beating for 3 min
mmm) Pipe the mixture onto a baking tray using a piping bag
nnn) Dry for one hour at 100° C. fan oven
ooo) Due to its very good whipping properties, the potato protein according to the invention is very well suited as a vegan chicken protein substitute.
ppp) Similarly, the meringue can also be produced by using the prepared protein solution directly without prior drying.

### Example 9—Protein-Enriched Pasta

| Ingredients | Concentration [%] |
|---|---|
| Clean label pea starch Empure Emjel 100 | 34 |
| Native pea starch | 30 |
| Potato protein according to the invention | 25 |
| Pea fiber Emfibre EF 200 | 10 |
| Guar gum | 1 |

qqq) Mixing all dry ingredients well
rrr) Add approx. 450-530 g of cold tap water per 1000 g of dry ingredients
sss) Mixing/kneading with the Häussler PN 300 VXS noodle machine for approx. 15 minutes
ttt) Compressing and shaping with a single screw extruder
uuu) Drying to a humidity content <13% by weight

vvv) By using the potato protein according to the invention, it was possible to produce a protein-enriched and gluten-free pasta.

### Example 10-Vegan Burgers

| Ingredients | Concentration [%] |
|---|---|
| Water | 63.0 |
| TVP (consisting of 90% by weight pea | 17.0 |
| protein Empro E 86 HV and 10% by weight | |
| potato fiber Emfibre KF 500) | |
| Oil | 10.0 |
| Potato protein according to the invention | 5.0 |
| Methyl cellulose | 1.5 |
| Guar gum | 1.5 |
| Psyllium husks | 1.0 |
| Clean label pea starch Empure ES 300 | 0.8 |
| Modified potato starch Emfix K 02 (E 1450) | 0.2 |
| Flavorings and colorings | To taste |

www) Allow TVP to rehydrate for 15 min
xxx) Shred the mixture to the desired particle size
yyy) Add all dry ingredients and emulsify with the oil
zzz) Shaping the burger patties

aaaa) The potato protein according to the invention formed a very solid gel directly when heated. This process could already be observed at relatively low temperatures, i.e. when frying the patties, whereas with many other proteins this can only be observed when cooling and also at higher temperatures. As a result, the use of the protein according to the invention led to a significant improvement in product binding, firmness and the meat-like mouthfeel. The gel formation of the potato protein according to the invention is irreversible.

bbbb) The produced protein solution of the potato protein according to the invention, without the drying step, can be used to produce an emulsion for vegan burgers. Advantageously, the potato protein according to the invention thus acts as an emulsifier. The above-mentioned advantages of the dried potato protein according to the invention also apply to the protein in solution.

cccc) When using the fat/oil, the lipase activity of patatin described above must be taken into account at this point. Vegetable fats and oils proved to be particularly suitable for this application.

dddd) It is also possible to blanch the formed burgers in a convection oven before preparation (50% humidity, 100° C., 15 min), which results in optimum texturing and development of the flavor profile.

### Example 11: Textured-Vegetable-Protein (TVP)

| Ingredients | Concentration [%] |
|---|---|
| Pea protein (Empro E 86 HV) | 45.0 |
| Potato protein according to the invention | 45.0 |
| Potato fiber (Emfibre KF 500) | 5.0 |
| Native potato starch | 5.0 |

eeee) Mixing the ingredients
ffff) Extruding the mixture using a Theysohn laboratory extruder (TSK 30) with a ratio of dry mixture:water of 98.5:1.5.
gggg) Drying the TVPs twice using the Pavan fluidized bed dryer (Mod. Tabatto Da Laboratorio) at 110° C.

hhhh) The potato protein according to the invention improved the stability of the TVPs (texturized vegetable proteins) after rehydration so that they did not become mushy. In addition, the use of the potato protein according to the invention improved the texture of the TVPs, which became firmer, more fibrous, more watery and thus more meat-like, which can be attributed to the excellent gelling properties of the potato protein according to the invention.

iiii) In the field of texturized vegetarian proteins (TVP), the solution of the potato protein according to the invention can be used in combination with a dry material mixture of dried protein according to the invention and possibly other ingredients (e.g. flours, starches or fibres). The advantages are similar to the use of dried protein.

### Example 12: Ready-to-shake Drink (RTS-Drink)

| Ingredients | Concentration [%] |
|---|---|
| Potato protein according to the invention | 94.47 |
| Deoiled cocoa powder | 2.70 |
| Cookie flavor | 1.50 |
| Chocolate flavor | 0.50 |
| Aspartame | 0.10 |
| Xanthan gum | 0.08 |

About 23.0 g Protein/Serving (30 g)

jjjj) Mix all the dry ingredients together.
kkkk) Pour about 300 mL of water or vegan milk into a protein shaker and add 30 g of the protein mixture.
llll) Mix all the ingredients by shaking for about 20 seconds.

mmmm) The use of the potato protein according to the invention resulted in the formation of a stable foam and a stable solution. The potato protein showed good solubility, resulting in a creamy mouthfeel. Although no fat, which should be avoided from a nutritional point of view, was added, the potato protein according to the invention resulted in a creamy mouthfeel.

nnnn) Although the invention has been explained in more detail with reference to examples of embodiments, it is obvious to a person skilled in the art that a wide variety of further embodiments are possible, wherein the scope of protection is determined solely by the claims.

1-17. (canceled)

18. A method for the preparation of a native, functional potato protein with a molecular weight of 10-120 kDa (according to SDS-Page primary structure), comprising the steps:
providing chopped, cleaned potato pieces under oxidation protection;
mechanically separating the solids from the chopped, cleaned potato pieces to produce potato amniotic fluid and solids;
adjusting the pH value of the potato amniotic fluid to a pH value between 6 and 9;
separating insoluble components and suspended solids by centrifugal separation,
ultrafiltration of the pH-adjusted potato amniotic fluid;
diafiltration of the ultrafiltration retentate with water to reduce the electrical conductivity of the retentate solution by 50-90%, with fully deionized water, distilled water, process water, or tap or process water while retaining the potato protein solution;
optionally, drying the potato protein solution and, optionally, sterilizing.

19. The method according to claim 18, wherein the pH value is adjusted after the mechanical solid/liquid separation to a pH value of 6-9.

20. The method according to claim 18, wherein drying is selected from spray drying, lyophilization, vacuum drying, freeze drying.

21. The method according to claim 18, wherein the ultrafiltration membrane is a plastic or ceramic membrane with a cut-off of 3-150 kDa.

22. The method according to claim 18, wherein by treating the protein solution optionally before or after ultrafiltration with adsorbents selected from activated carbon, bentonites, polymeric adsorbents, and derivatives thereof.

23. The method according to claim 18, wherein the ultrafiltration retentate is gently sterilized.

24. The method according to claim 23, wherein the sterilization is selected from pasteurization with microwaves or HTST pasteurization (High Temperature Short Time), PEF sterilization (Pulsed Electric Fields), HPP pasteurization (High Pressure Pasteurization).

25. The method according to claim 18, wherein the water used for diafiltration has a conductivity of 5 μS/cm-5000 μS/cm.

26. The method according to claim 18, wherein the pH value of the potato amniotic fluid is adjusted to a pH value between 9-7.5.

27. The method according to claim 18, wherein the diafiltration of the ultrafiltration retentate with water reduces the electrical conductivity of the retentate solution by 75-85%

28. A potato protein produced by:
providing chopped, cleaned potato parts in the form of potato mash under oxidation protection, mechanical separation of the potato mash into solid and liquid phase (potato amniotic fluid);
adjusting the pH value to 6-9,
separating insoluble components and suspended solids by centrifugal separation;
ultrafiltration of the liquid phase with membranes with a cut-off of 3-150 kDa,
diafiltration of the ultrafiltration retentate with fully deionized water, distilled water, process water or tap or process water up to a reduction in conductivity of 50-90%,
removing antinutritive substances using adsorption techniques;
optionally, sterilizing and
optionally, drying the protein solution.

29. A potato protein according to claim 28, comprising
a) a protein content of at least 84% absolute dry according to Kjeldahl,
b) a product solubility/protein solubility of the product based on 10% by weight humidity of dried protein in tap water from 75-100% and
c) an ash content of max. 3% by weight.

30. The potato protein according to claim 28, comprising a humidity content of max. 10% by weight after drying.

31. The potato protein according to claim 28, comprising a molecular weight of 10-120 kDa (according to SDS-Page primary structure).

32. The potato protein according to claim 28 comprising three main fractions, a first main fraction having a molecular weight of 10 to 20 kDa, a second main fraction having a molecular weight of between 25 and 40 kDa and a third main fraction having a molecular weight of about 66 kDa according to SDS-Page primary structure.

33. The potato protein according to claim 28, wherein the potato protein is a component of a food or food additive, a dietary food or food additive for human or animal consumption.

34. The potato protein according to claim 28, wherein the potato protein is a component of an adhesive.

35. The potato protein according to claim 28, wherein the potato protein is a component of a mixture with other vegetable proteins,

36. The potato protein according to claim 28, wherein the potato protein is a component of a mixture with animal proteins.

37. The potato protein according to claim 28, wherein the potato protein is a component of a mixture with at least one legume protein from the group consisting of pea protein, bean protein, field bean protein, lentil protein, soy protein, lupin protein, mung bean protein, or mixtures thereof.
